(19) United States
(12) Patent Application Publication
(10) Pub. No.: US 2016/0374644 A1
(43) Pub. Date: Dec. 29, 2016

(54) Ultrasonic Guidance of a Probe with Respect to Anatomical Features

(71) Applicant: Rivanna Medical LLC, Charlottesville, VA (US)

(72) Inventors: Frank William Mauldin, JR., Charlottesville, VA (US); Kevin Owen, Crozet, VA (US); Adam Dixon, Charlottesville, VA (US)

(21) Appl. No.: 15/152,840

(22) Filed: May 12, 2016

Related U.S. Application Data
(60) Provisional application No. 62/184,594, filed on Jun. 25, 2015.

(51) Int. Cl. A61B 8/00; A61B 8/08

(52) U.S. Cl. CPC A61B 8/4245; A61B 8/463; A61B 8/5207; A61B 8/469; A61B 8/0841; A61B 8/4444

(57) ABSTRACT

Systems and methods for probe insertion using feedback from ultrasound guidance using anatomical features. The present disclosure is directed to ultrasound imaging for the generation of ultrasound images of anatomical features such as bone and/or visualizing ultrasound images of anatomical features in a subject being imaged. Specifically, the present invention pertains to real-time feedback using graphical user interface and ultrasonic imaging for the purpose of probe insertion. Probe insertion can either be idealistically displayed or physically guided with varying degrees of freedom for augmented accuracy and mitigating failure rates.

### RELATED APPLICATIONS

The present application claims the benefit and priority of U.S. Provisional Application No. 62/184,594, entitled “Ultrasonic Guidance of a Probe with Respect to Anatomical Features,” filed on Jun. 25, 2015, which is hereby incorporated by reference.

### STATEMENT OF FEDERALLY SPONSORED RESEARCH

This invention was sponsored at least in part using U.S. Government support under award number R44EB015232 from the National Institute of Biomedical Imaging and Bioengineering of the National Institutes of Health, and award number 1329651 from the National Science Foundation. The U.S. Government may thus have certain rights in this invention.

### TECHNICAL FIELD

The present disclosure is directed to ultrasound imaging and systems and methods for ultrasonic image acquisition and generation. Aspects of the disclosure relate to generating ultrasound images of bone and/or visualizing ultrasound images of bone in a subject being imaged. Specifically, the present invention pertains to automated detection of target anatomy and real-time feedback using graphical user interface with ultrasonic imaging for the purpose of probe insertion.

### BACKGROUND

Various medical procedures comprise penetrating the skin with a probe, such as a needle or a catheter. For example, spinal anesthesia or a spinal diagnostic procedure can include percutaneous delivery of anesthetic to an epidural location or sampling of spinal fluid. Such spinal anesthesia or spinal diagnostic procedures generally include penetrating the ligamentum flavum, a ligament between the spinous processes lateral to the dura. Generally, a desired final needle position during epidural placement is lateral the dura, while in a spinal tap, the dura is penetrated in order to obtain fluid from the spinal cavity.

Spinal taps have several important clinical applications including sampling cerebral spinal fluid (CSF), administering chemotherapy or other drugs directly into the spinal cavity, or relieving pressure in the spinal cavity for cardiac procedures. Sampling of CSF can also be necessary to quickly diagnose various diseases such as meningitis. Other procedures can similarly include penetrating the skin with a probe, such as paravertebral somatic nerve blockade (PVB).

Neuroaxial anesthesia blocks (e.g., epidural anesthesia or spinal anesthesia blocks) and related spinal anesthesia procedures are presently performed in millions of procedures per year in U.S. hospitals. Numerous clinical indications for such procedures include anesthesia during pregnancy, chronic pain, or hip or knee replacement surgery.

Given the importance of probe placement due to its sensitive location, imaging can be used to ameliorate probe guidance. In one approach, fluoroscopy can be used to guide spinal needle placement with high success. However, the risk of ionizing radiation, in addition to high cost and lack of portability of fluoroscopy equipment, make fluoroscopy an unattractive option for some high-volume procedures.

Other x-ray based medical imaging techniques can also be effective but suffer from similar risks and disadvantages. For example, computed tomography (CT) and 2-dimensional x-ray projection are frequently used as imaging modalities for bone imaging. Unfortunately, ionizing radiation exposure to patients and caregivers from such medical imaging has increased dramatically in past decades (estimated at a several fold increase in recent decades). The cumulative effect of such radiation dosages has been linked to increased risk of cancer.

During a medical procedure, a probe insertion can sometimes be accomplished without requiring medical imaging (i.e., using an unguided technique). The technique without medical imaging is called the “blind approach.” In the spinal anesthesia application, this comprises needle insertion after locating spinal bone landmarks using manual palpation. However, such unguided techniques can sometimes fail. Unguided spinal anesthesia or spinal diagnostic procedure failures typically occur in the elderly or severely/morbidly obese. Reasons for failure in unguided procedures include incorrect needle insertion location or use of an incorrect needle angle during penetration.

Consequently, in a spinal anesthesia or a spinal diagnostic procedure, failure can prevent access to the spinal cavity or preclude placement of a needle or catheter lateral the dura for administration of an epidural. Failure rates for blind approaches have been historically cited in about half of the patient populations exhibiting landmarks that are absent, indistinct, or distorted.

A significant and growing population segment exhibiting these characteristics is the obese that currently make up about a third of the total U.S. population but represent a disproportionately high blind failure rate. That is, failure of unguided procedures can occur at rates as high as three quarters of cases involving obese patients. Such failures can increase healthcare costs, such as those arising from complications requiring additional treatment.

In the severely/morbidly obese, such failure can occur because anatomical landmarks (e.g., spine) cannot be reliably palpated due to thick layers of fatty tissue between the landmarks and the skin. Failures generally result in multiple needle sticks, which are correlated with poor health outcomes such as an increased risk of spinal headache or hematoma. In addition, other serious complications can occur from failed neuroaxial anesthesia including back pain or vascular puncture, as well as more severe complications including pleural puncture, pneumothorax, or paralysis. Such complications can include spinal headaches, back pain, paraparesis, spinal hematoma, nerve palsy, spinal tumor formation, or one or more other complications.

Generally, when the unguided approach fails, the clinical procedure includes using fluoroscopy or other guided procedures to assist in probe placement. Medical ultrasound may be used as an alternative to x-ray for bone imaging. However, even though they do not pose the risk of ionizing radiation, conventional ultrasound systems are limited in their application. Ultrasound systems currently in use are generally large, complicated, expensive, and require specialized training to operate. However, failure rates using ultrasound can still remain high, and the success of ultrasonic techniques has generally been highly dependent on user familiarity with ultrasonography. Also, traditional ultrasound equipment is heavy and bulky thus making it difficult to use with patients.

Therefore, there exists a need for user-friendly guidance system for probe insertion using non-ionizing ultrasonic imaging.

### SUMMARY

Example embodiments described herein have innovative features, no single one of which is indispensable or solely responsible for their desirable attributes. The following description and drawings set forth certain illustrative implementations of the disclosure in detail, which are indicative of several exemplary ways in which the various principles of the disclosure may be carried out. The illustrative examples, however, are not exhaustive of the many possible embodiments of the disclosure. Without limiting the scope of the claims, some of the advantageous features will now be summarized. Other objects, advantages and novel features of the disclosure will be set forth in the following detailed description of the disclosure when considered in conjunction with the drawings, which are intended to illustrate, not limit, the invention.

An aspect of the invention is directed to an ultrasound imaging method. The method includes, in a probe guidance system comprising a processor and a probe guide having a specified path along which to insert a probe, transmitting one or more ultrasound signals from one or more transducers in the probe guidance system. The method also includes obtaining ultrasound data generated based, at least in part, on one or more ultrasound signals from an imaged region of a subject. The method also includes selecting a target anatomy associated with the imaged region based at least in part on the generated ultrasound data. The method also includes displaying an ultrasound image of the subject at least in part by combining the ultrasound data and the selected target anatomy. The method also includes determining a location of the imaged region relative to the target anatomy and the one or more transducers. The method also includes calculating a projected probe path of the probe, the projected probe path indicative of an actual path to be taken by the probe when the probe is inserted through the probe guide. The method also includes generating a graphic indicator including generating a visible representation of said projected probe path, the visible representation of the projected probe path displayed with respect to said target anatomy.

In some embodiments, the projected probe path includes a projected needle path. The method can include providing feedback in a loop when the probe guidance system determines that the projected probe path and the target anatomy are not collinear. The method can also include displaying a directional indicator to indicate a direction to translate the one or more transducers to align the projected probe path with the target anatomy. The method can also include comprising displaying a rotational indicator to indicate a motion necessary to align the projected probe path with the target anatomy.

In some embodiments, the method includes calculating an ideal probe path, the ideal probe path coaxially intersecting the target anatomy. The method can also include restricting the ideal probe path to potential probe paths that exhibit one or more physical pivot points by which an angle of said probe guide can rotate. The method can also include restricting the ideal probe path to potential probe paths that exhibit one or more virtual pivot points by which an angle of said probe guide can rotate. The method can also include calculating and displaying one or more displayed needle paths on a graphical user interface and comprising a user selecting and executing one of the displayed needle paths via interaction with a graphical user interface.

Another aspect of the invention is directed to a probe guidance system. The system includes a user interface having a display with one or more symbolic indicators. The system also includes one or more ultrasonic transducers of an ultrasonic imaging unit configured and adapted to transmit and receive signals based at least in part on a target anatomy. The system also includes a probe guide having a specified path along which to insert a probe. The system also includes a processor for (a) determining a location of the target anatomy relative to the ultrasound imaging system and (b) calculating a direction to translate or rotate the one or more transducers to align (x) a projected probe path of the probe, the projected probe path indicative of an actual path to be taken by the probe when the probe is inserted through the probe guide, with (y) the target anatomy.

In some embodiments, the displayed symbolic indicator represents the direction for a user to translate or rotate the one or more transducers. In some embodiments, the probe guide provides a variable rotational orientation relative to a surface of a patient.

The system can include an integrated, real-time needle detection device. In some embodiments, the integrated, real-time needle detection device is optical. In some embodiments, the integrated, real-time needle detection device includes a piezoelectric element.

In some embodiments, the processor calculates an actual probe angle and determines a probe angle adjustment needed to align the projected probe path with the target anatomy. The display can include a touch-pad adapted and configured to accept user input to identify said target anatomy.

In some embodiments, at least a portion of the probe guide is rotatable about a pivot point. The probe guide can include a guide spool that defines the specified path along which to insert the probe, the pivot point on the guide spool. The system can include a compression mechanism that contacts the guide spool to retain the guide spool at a desired orientation.

This overview is intended to provide an overview of subject matter of the present patent application. It is not intended to provide an exclusive or exhaustive explanation of the invention. Further limitations and disadvantages of conventional and traditional approaches will become apparent to one of skill in the art, through comparison of such systems with some aspects of the present invention as set forth in the remainder of the present application with reference to the drawings.

### BRIEF DESCRIPTION OF THE DRAWINGS

For a fuller understanding of the nature and advantages of the present invention, reference is made to the following detailed description of preferred embodiments and in connection with the accompanying drawings, in which:

FIG. 1 is a block diagram of an exemplary apparatus that may include at least one ultrasound transducer and at least one processor configured to perform anatomical imaging, the output of which may be rendered to the apparatus display, in accordance with some embodiments of the disclosure provided herein;

FIG. 2 is a top-down view of an exemplary, portable 2D ultrasound imager with graphical user interface feedback and probe guide together with a 3D model of at least a portion of the imaged area, in accordance with some embodiments of the disclosure provided herein;

FIG. 3 is a side view of an exemplary, portable 2D ultrasound imager with graphical user interface feedback and probe guide together with a 3D model of at least a portion of the imaged area, in accordance with some embodiments of the disclosure provided herein;

FIG. 4 is a side view of an exemplary, portable 2D ultrasound imager with graphical user interface feedback and probe guide together with a 3D model of at least a portion of the imaged area, in accordance with an alternative embodiment of the disclosure provided herein;

FIG. 5 is a diagram illustrating an exemplary probe guide with a rotational degree of freedom, in accordance with some embodiments of the disclosure provided herein;

FIG. 6 is a flowchart of an illustrative process of directing a probe in fixed guide to a predetermined, anatomical location based at least in part on ultrasonic imaging, in accordance with some embodiments of the disclosure provided herein;

FIG. 7 is a flowchart of an illustrative process of directing a probe in fixed guide to a user-identified anatomical location based at least in part on ultrasonic imaging, in accordance with some embodiments of the disclosure provided herein;

FIG. 8 depicts an exemplary graphical user interface demonstrating probe directional location feedback and overlaid ultrasound image of target anatomy, in accordance with some embodiments of the disclosure provided herein;

FIG. 9 depicts an exemplary graphical user interface demonstrating probe rotational disposition and directional feedback and overlaid ultrasound image of target anatomy, in accordance with some embodiments of the disclosure provided herein;

FIG. 10 is a top-down view of a portable 2D ultrasound imager with graphical user interface feedback depicting exemplary probe insertion and guidance thereto, in accordance with some embodiments of the disclosure provided herein;

FIG. 11 is a flowchart of an exemplary procedure for directing a probe without a fixed guide to a user-identified anatomical location based at least in part on a generated ultrasonic image, in accordance with some embodiments of the disclosure provided herein;

FIG. 12 projects an isometric view of an exemplary virtual probe guide rotating about a fixed pivot axis in the image plane for use during device assisted guidance, in accordance with some embodiments of the disclosure provided herein;

FIG. 13 illustrates a side view of an exemplary virtual probe guide rotating about a fixed pivot axis in the image plane for use during device assisted guidance, in accordance with some embodiments of the disclosure provided herein;

FIG. 14 illustrates a top-down view of an exemplary virtual probe guide rotating about a fixed pivot axis in the image plane for use during device assisted guidance, in accordance with some embodiments of the disclosure provided herein;

FIG. 15 is a graphical abstraction of a side view of an exemplary virtual probe guide rotating about a fixed pivot axis in the image plane juxtaposed to a corresponding graphical user interface output with virtual state and disposition, in accordance with some embodiments of the disclosure provided herein;

FIG. 16 is a graphical abstraction of a side view of an exemplary virtual probe guide rotating about a fixed pivot axis in the image plane juxtaposed to a corresponding graphical user interface output with virtual state and disposition, in accordance with some embodiments of the disclosure provided herein;

FIG. 17 is a graphical abstraction of a side view of an exemplary virtual probe guide rotating about a fixed pivot axis in the image plane juxtaposed to a corresponding graphical user interface output with virtual state and disposition, in accordance with some embodiments of the disclosure provided herein;

FIG. 18 is a graphical abstraction of a side view of an exemplary virtual probe guide rotating about a fixed pivot axis in the image plane juxtaposed to a corresponding graphical user interface output with virtual state and disposition, in accordance with some embodiments of the disclosure provided herein;

FIG. 19 is a graphical abstraction of a side view of an exemplary virtual probe guide rotating about a fixed pivot axis in the image plane juxtaposed to a corresponding graphical user interface output with virtual state and disposition, in accordance with some embodiments of the disclosure provided herein;

FIG. 20 is a graphical abstraction of a side view of an exemplary virtual probe guide rotating about a fixed pivot axis in the image plane juxtaposed to a corresponding graphical user interface output with virtual state and disposition, in accordance with some embodiments of the disclosure provided herein;

FIG. 21 illustrated an exemplary handheld 2D ultrasound imager with graphical user interface feedback and non-affixed probe guide together with a 3D model of at least a portion of the imaged area, in accordance with some embodiments of the disclosure provided herein; and

FIG. 22 illustrated an exemplary portable 2D ultrasound imager coupled to external computational unit via data communication, in accordance with some embodiments of the disclosure provided herein.

### DETAILED DESCRIPTION

The following description and drawings set forth certain illustrative implementations of the disclosure in detail, which are indicative of several exemplary ways in which the various principles of the disclosure may be carried out. The illustrative examples, however, are not exhaustive of the many possible embodiments of the disclosure. Other objects, advantages and novel features of the disclosure are set forth in the proceeding in view of the drawings where applicable.

Embodiments of the proposed apparatus can enable more accurate puncture or probe insertion procedures by providing information to the user about a depth or location of bone with respect to the probe. Aspects of the present invention are directed to probe guidance and insertion based on sonographic imaging of anatomical features. The inventors have recognized that unguided needle insertion for medical procedures exhibit substantial failure rates, particularly in an increasing demographic of the population. Anatomical features cannot be accurately palpated in all patients. Imaging an area of a subject which circumscribes the procedural location and augmenting ultrasound images with automatic identification of target regions of tissue greatly improves the success of probe insertions and ease of use.

The inventors have also recognized that an ultrasound image may be easier to interpret if presented (e.g., to a user) with reference to an anatomical model of the structure being imaged. In an example, the structure being imaged includes bone or tissue lying in, near or between bone structures. Accordingly, some embodiments relate to visualizing ultrasound data by generating a visualization of a two-dimensional (2D) ultrasound image that includes a corresponding portion of a three-dimensional (3D) structure model. In certain applications, the structure of interest is a bone structure, such as the spinal bone anatomy. The corresponding portion of the 3D model (e.g., a 2D cross-section) may be identified at least in part by using a registration technique to register the 2D ultrasound image to the 3D model. The registration results may be used to identify the location(s) of one or more anatomical landmarks in the 2D ultrasound image and the generated visualization of the image may indicate one or more of the identified locations.

Aspects of the present invention disclose the generation of ultrasound images of needle targeted anatomy and/or visualizing ultrasound images in a subject for the purpose of real-time feedback using graphical user interface (GUI) and ultrasonic imaging for the purpose of probe insertion. In an application, the target anatomy is defined with respect to a bone structure, e.g., spinal vertebrae or other bone structure, as well as tissues in or between such bone structures. However, this is only one way to apply the present concepts, which can equally apply to other target regions. The present inventors have also recognized similar needs in other needle-guided applications such as joint injections and aspirations, vascular access, and biopsies. In such cases, medical imaging can be used to navigate a needle or probe to a target anatomy. Automation of the target anatomy and real-time guidance feedback, can make the medical imaging guidance easier to use.

The present inventors have also recognized that a portable apparatus can be less expensive than generally available B-mode imaging equipment. Also, incorporation of display into a hand-held device can be manufactured to provide an intuitive or easy-to-understand indication of a target anatomy location or depth, as compared to a B-mode sonogram that can be difficult to interpret. Use of the hand-held apparatus can also reduce medical costs because the hand-held apparatus can be used for guided probe insertion or anatomical location thereby reducing likelihood of failure or complication during a probe insertion. The apparatus can also be operated without extensive training in ultrasonography.

Such a hand-held apparatus can be simpler to operate than generally available ultrasound imaging equipment. For example, information provided by a hand-held apparatus can be less resource consuming and simpler to interpret—in contrast to generally available B-mode ultrasonic imaging equipment. The present disclosure contemplates the fabrication of a novel portable device with a graphical user interface (GUI) for giving user feedback of probe insertion, depth, disposition, location and orientation, as well as practical methods for the application thereof and remedying these and/or other associated problems.

Aspects of the technology described herein are explained in the context of spinal anesthesia guidance, but it should be appreciated that the technology described herein is useful for and may be applied in many other settings. For example, the technology described herein may be used for other clinical applications where ultrasound is used to guide a needle or probe to a target anatomy including, but not limited to, guiding of orthopedic joint injections, vascular access, and biopsies.

In some embodiments, a method for performing ultrasound imaging with a graphical user interface (GUI) is provided. The method may comprise building a 3D model based on patient anatomical features in conjunction with known models and/or predetermined patient models such as those derived from a priori MRIs or CAT scans, at least in part. The inventors also recognize the efficacy of displaying the model relative to the probe guided device in a simple, easy to understand manner—particularly, with comprehensive, globally-recognizable graphical symbols and visual cues. The present inventors recognize that detecting anatomical targets can be performed through other methods besides model fitting, including various feature detection algorithms known to those of skill in the art, such as shape models or Hough transform.

In some embodiments, the method comprises registering at least one 2D ultrasound image to a 3D model of a region comprising bone; and producing a 2D and/or 3D visualization of the region comprising bone wherein the visualization is derived, at least in part, from the registration of the at least one 2D ultrasound image to the 3D model of the spine. Registration can be performed by ultrasonically surveying a substantial portion of a patient's spine; accessing existing libraries and analyzing its contents with respect to pattern matching to the patient's sonogram; and/or loading 3D model from a previously performed scan (e.g., MRI, etc.) of the patient.

The aspects and embodiments described above, as well as additional aspects and embodiments, are described further below. These aspects and/or embodiments may be used individually, all together, or in any combination of two or more, as the technology described herein is not limited in this respect.

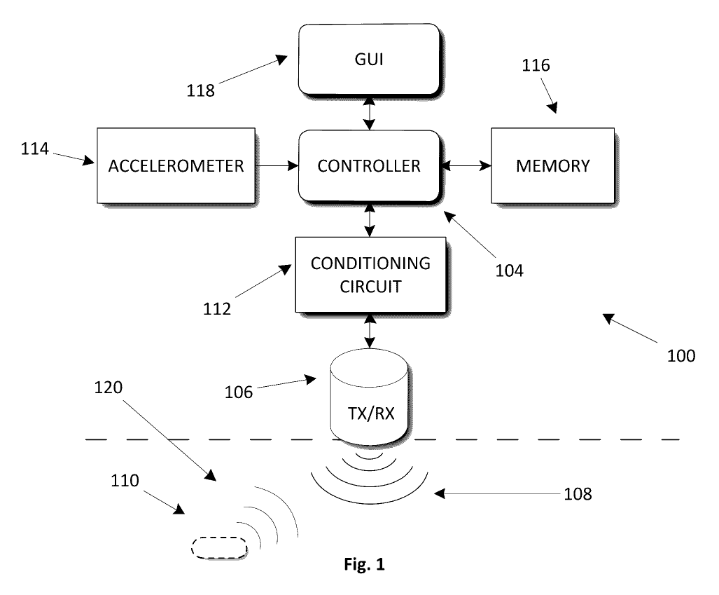

FIG. 1 illustrates an example of an apparatus 100 that may be used for generating and/or displaying ultrasound images. As shown, apparatus 100 comprises at least one processor control circuit 104, at least one ultrasound transducer 106, at least one ultrasound signal conditioning circuit 112, at least one motion sensor (accelerometer) 114, at least one memory circuit 116, and graphical user interface/display 118. The one or more ultrasound transducers 106 may be configured to generate ultrasonic energy 108 to be directed at a target tissue structure 110 within a subject being imaged (e.g., the ultrasound transducers 106 may be configured to insonify one or more regions of interest within the subject).

Some of the ultrasonic energy 108 may be reflected 120 by the target tissue structure 110, and at least some of the reflected ultrasonic energy may be received by the ultrasound transducers 106. In some embodiments, the at least one ultrasonic transducer 106 may form a portion of an ultrasonic transducer array, which may be placed in contact with a surface (e.g., skin) of a subject being imaged. In some embodiments, ultrasonic energy reflected 120 by the subject being imaged may be received by ultrasonic transducer(s) 106 and/or by one or more other ultrasonic transducers, such as one or more ultrasonic transducers that are part of a transducer array. The ultrasonic transducer(s) that receive the reflected ultrasonic energy may be geometrically arranged in any suitable way (e.g., as an annular array, a piston array, a linear array, a two-dimensional array) or in any other suitable way, as aspects of the disclosure provided herein are not limited in this respect.

As illustrated in FIG. 1, ultrasonic transducer(s) 106 may be coupled to the ultrasonic signal conditioning circuit 112, which is shown as being coupled to circuits in apparatus 100. The ultrasonic signal conditioning circuit 112 may include various types of circuitry for use in connection with ultrasound imaging such as beam-forming circuitry, for example. As other examples, the ultrasonic signal conditioning circuit may comprise circuitry configured to amplify, phase-shift, time-gate, filter, and/or otherwise condition received ultrasonic information (e.g., echo information), such as provided to the processor circuit 104.

In some embodiments, the receive path from each transducer element from part of a transducer array, such as an array including the first ultrasonic transducer 106, may include one or more of a low noise amplifier, a main-stage amplifier, a band-pass filter, a low-pass filter, and an analog-to-digital converter. In some embodiments, one or more signal conditioning steps may be performed digitally, for example by using the processor controller circuit 104.

In some embodiments, the apparatus 100 may be configured to obtain ultrasonic echo information corresponding to one or more planes perpendicular to the surface of an array of ultrasound transducers (e.g., to provide “B-mode” imaging information). For example, the apparatus 100 may be configured to obtain information corresponding to one or more planes parallel to the surface of an array of ultrasound transducers (e.g., to provide a “C-mode” ultrasound image of loci in a plane parallel to the surface of the transducer array at a specified depth within the tissue of the subject). In an example where more than one plane is collected, a three-dimensional set of ultrasonic echo information may be collected.

In some embodiments, the processor controller circuit 104 may be coupled to one or more non-transitory computer-readable media, such as the memory circuit 116, a disk, or one or more other memory technology or storage devices. In some embodiments, a combination of one or more of the first ultrasonic transducer 106, the signal conditioning circuit 112, the processor controller circuit 104, the memory circuit 116, and graphical user interface (display) 118 may be included as a portion of an ultrasound imaging apparatus. The ultrasound imaging apparatus may include one or more ultrasound transducers 106 configured to obtain depth information via reflections of ultrasonic energy from an echogenic target tissue structure 110, which may be a bone target, blood vessel, lesion, or other anatomical target.

In an example, the processor controller circuit 104 (or one or more other processor circuits) may be communicatively coupled to one or more of a user input device, such as a graphical user interface 118. In other embodiments, the user input device may include one or more of a keypad, a keyboard (e.g., located near or on a portion of ultrasound scanning assembly, or included as a portion of a workstation configured to present or manipulate ultrasound imaging information), a mouse, a rotary control (e.g., a knob or rotary encoder), a soft-key touchscreen aligned with a portion of a display, and/or one or more other controls of any suitable type.

In some embodiments, the processor controller circuit 104 may be configured to perform model registration-based imaging and presenting the constructed image or images to the user via the GUI 118. For example, a simultaneous 2D/3D display may be presented to the user via the GUI 118.

In some embodiments, ultrasonic energy reflected 120 from target tissue 110 may be obtained or sampled after signal conditioning through the ultrasound signal conditional circuit 112 as the apparatus 100 is swept or moved across a range of locations along the subject surface (e.g., skin). A composite may be constructed such as using information about the location of at least the transducer 106 of apparatus 100 (or the entire apparatus), such as provided by the motion sensor 114, and information about reflected ultrasonic energy obtained by the ultrasonic transducer 106.

Motion sensor or accelerometer 114 may be any suitable type of sensor configured to obtain information about motion of the subject being imaged (e.g., position information, velocity information, acceleration information, poses information, etc.). For example, the motion sensor 114 may comprise one or more accelerometers configured to sense acceleration along one or more axes. As another example, the motion sensor 114 may comprise one or more optical sensors. The motion sensor 114 may be configured to use one or more other techniques to sense relative motion and/or absolute position of the apparatus 100, such as using electromagnetic, magnetic, optical, or acoustic techniques, or a gyroscope, such as independently of the received ultrasound imaging information (e.g., without requiring motion tracking based on the position of imaged objects determined according to received ultrasonic information).

Information from the motion sensor 114 and ultrasonic energy obtained by the ultrasonic transducer 104 may be sent to the processor controller circuit 104. The processor controller circuit 104 may be configured to determine motion or positional information of at least the transducer of apparatus 100 using processes described in further examples below. The motion or positional information may be used to carry out model registration-based imaging or freehand 3D imaging.

Other techniques may include using one or more transducers that may be mechanically scanned, such as to provide imaging information similar to the information provided by a two-dimensional array, but without requiring the user to manually reposition the apparatus 100 during a medical procedure. The apparatus 100 may be small and portable, such that a user (e.g., a physician or nurse) may easily transport it throughout healthcare facilities or it may be a traditional cart-based ultrasound apparatus.

In some embodiments, apparatus 100 may provide imaging using non-ionizing energy, it may be safe, portable, low cost, and may provide an apparatus or technique to align a location or insertion angle of a probe to reach a desired target depth or anatomical location. Examples of the model registration-based process described below are focused on spinal anesthesia clinical procedures whereby a healthcare professional inserts a probe in or around the spinal bone anatomy to deliver anesthetics.

In this instance the model registration-based process uses a 3D model of the spinal bone anatomy. However, the apparatus and methods described herein are not limited to being used for imaging of the spine and may be used to image any suitable target anatomy such as bone joints, blood vessels, nerve bundles, nodules, cysts, or lesions. In addition, apparatus 100 may be employed in clinical diagnostic or interventional procedures such as orthopedic joint injections, lumbar punctures, bone fracture diagnosis, and/or guidance of orthopedic surgery.

It should be appreciated that the apparatus 100 described with reference to FIG. 1 is an illustrative and non-limiting example of an apparatus configured to perform ultrasound imaging in accordance with embodiments of the disclosure provided herein. Many variations of apparatus 100 are possible. For example, in some embodiments, an ultrasound imaging apparatus may comprise one or more transducers for generating ultrasonic energy and circuitry to receive and process energy reflected by a target being imaged to generate one or more ultrasound images of the subject, but may not comprise a display to display the images. Instead, in some embodiments, an ultrasound imaging apparatus may be configured to generate one or more ultrasound images and may be coupled to one or more external displays to present the generated ultrasound images to one or more users.

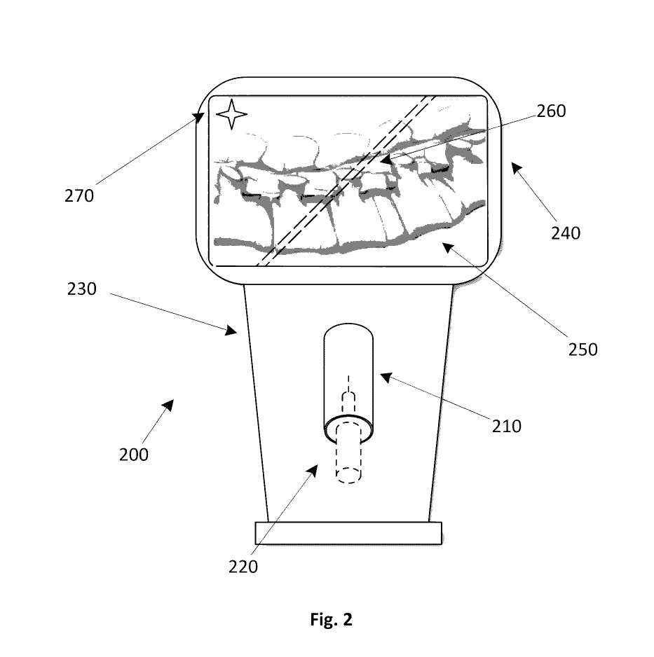

FIG. 2 is a top-down view of an exemplary, portable 2D ultrasound system 200 with graphical user interface feedback 270 and probe guide 210. In an aspect, the system includes an automated anatomy detector, which may employ anatomical imaging of a variety (or a plurality) of imaging modalities. In another aspect, this system is used together with a model of at least a portion of the imaged target area 250, which may be a 3-dimensional (3D) model or other suitable model, however this is not required for the operation of the system. In one embodiment, ultrasound system 200 automates identification of target anatomy 250, provides an indication of the target mid-line and depth 260, and provides indication of transducer motion required to align target anatomy with a desired probe path. Those skilled in the art will appreciate that the present concepts are applicable to automated anatomy detection generally and may employ one or more imaging modalities.

Identification of a target anatomy 250 with the aid of user input via a touchscreen 240 or other method provides an indication of the target mid-line and depth 260 then indicates how to move the transducer to align the target with the probe 220 path. Ultrasonic system 200 continually tracks the target with each new frame with continuous feedback on position relative to probe 220 path. In one or more embodiments, the probe is a needle. In other embodiments, the probe is a catheter or other similar device, which is not beyond the scope of the present invention.

Automatic identification of the target anatomy 250 can be achieved through a variety of methods. In one embodiment, the target anatomy 250 can be detected via user interacts with the touch screen 240 image feature. Once the target anatomy 250 is identified by the user, the ultrasound system 200 can then track the feature as it changes position or orientation as the position of the transducer, relative to the target anatomy 250, changes.

Tracking of the target anatomy 250 feature can be achieved through a variety of methods known to those of ordinary skill in the art. Such methods include template matching techniques—e.g. normalized cross-correlation, sum of absolute differences, etc. Other methods include model fitting such as using adaptive shape models. The shape model can be formed from a priori knowledge of the target anatomy or adaptively from the image region indicated by the user.

In one embodiment, a model based technique can be used to automatically detect a target anatomy. In this approach, the model is formed a priori based on knowledge of the desired target anatomy 250. In an example of a model-based technique to detect a target anatomy, the approach would not require user input. However user input could be used to help guide the search process. For example, if the user indicates a particular location of the image, optionally using a user interface, then the algorithm can bias the search result to that location.

In another embodiment, detection of blood flow or other functional measurements can be used to identify a target. For example, if the target anatomy is a blood vessel, then the target location can be calculated from a blood flow image. Specifically, the centroid location of the blood flow can be calculated from all image locations where blood flow presence was detected. Image locations with blood flow presence can be measured using standard methods such as color Doppler, B-flow, pulse wave Doppler, or power Doppler.

In other embodiments, a Hough transform, shape model, or template matching scheme can identify locations in the image exhibiting a representative shape or spatially varying intensity. The centroid of the various locations can be computed. Multiple potential targets can be presented to the user for selection via a graphical user interface input, such as via a touchscreen.

In the present embodiment exemplified in FIG. 2, the device comprises a needle guide 210 with a fixed path disposed on or in a handle 230 so that the overall device 200 is hand-held. The device 200 may be battery operated and conveniently portable and placed in a practitioner's pocket, in a travel pouch, case or similar housing. In use, a clinical practitioner may deploy the device (seen from above from the practitioner's point of view) onto a surface of a patient's body, e.g., the skin above the patient's spine region. The guide 210 provides a path that would be followed by a rigid structure or probe 220 inserted through the guide, which can be displayed (260) on the display screen overlaying the ultrasound image of the target anatomy 250. Those skilled in the art will appreciate that the present concepts can apply to inserting a needle into the patient's body and can also apply to insertion of other elongated probes, catheters and so on into the patient with respect to the patient's anatomy, e.g., bone anatomy.

The ultrasound image can be any mode of ultrasound imaging and can be 2D or 3D. In some embodiments, the ultrasound system displays B-scan sonographs. Color sonograms, which may include post processed and enhanced images to aid the intended procedure may also be employed as would be appreciated by one of skill in the art upon review of the present disclosure. C-scan sonography is also within the scope of the present invention. Ultrasound imaging arrays and transducers of any suitable design and configuration may be employed. The present disclosure is not limited to transducers or transducer arrays of any given geometry, size or frequency range. But ultrasound in the high kilo Hertz to low or mid mega Hertz range can be used in some embodiments.

As mentioned above, the ultrasound needle guidance and imaging system 200 of the present exemplary embodiment can be handheld as is demonstrated. However, compact and cart-based systems are also easily incorporated, which will be discussed later in the disclosure.

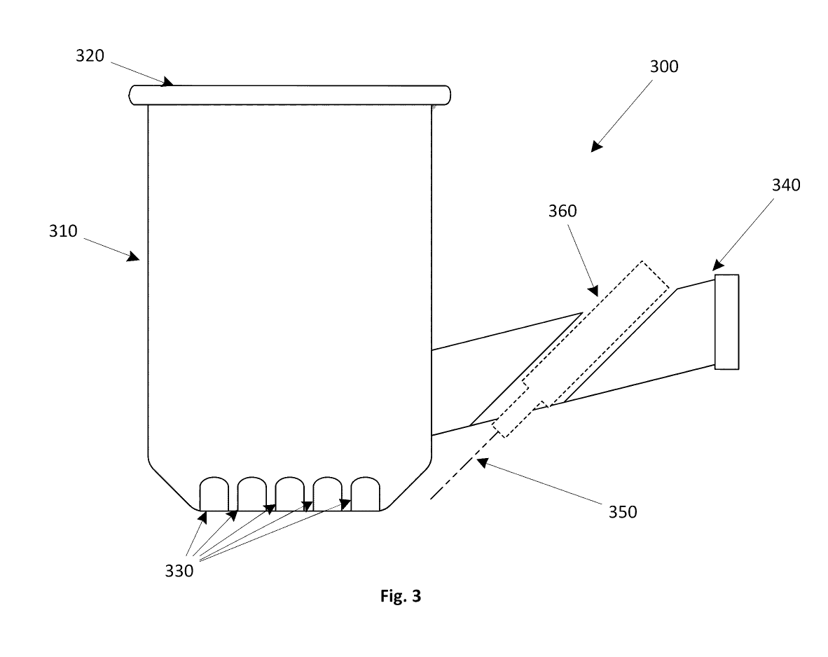

FIG. 3 is a side view of an exemplary, portable ultrasound imaging and probe guidance system 300 that includes a body 310, which may be hand-held, a graphical user interface 320, and probe guide 340 through which a needle assembly 360 can be inserted for guidance. Ultrasonic system body 310 comprises one or more ultrasound imaging transducers 330 at its lower end that contacts a patient's body proximal to a region of interest, for example, the transducers 330 can be placed on the patient's skin (coupled using ultrasonic coupling gel) to image the anatomical structures below the probe. Probe guide 340 is disposed angularly for needle 350 for non-orthogonal insertion. However, the angle of probe guide 340, and hence the angle of needle assembly 360 with respect to body 310 need not be fixed.

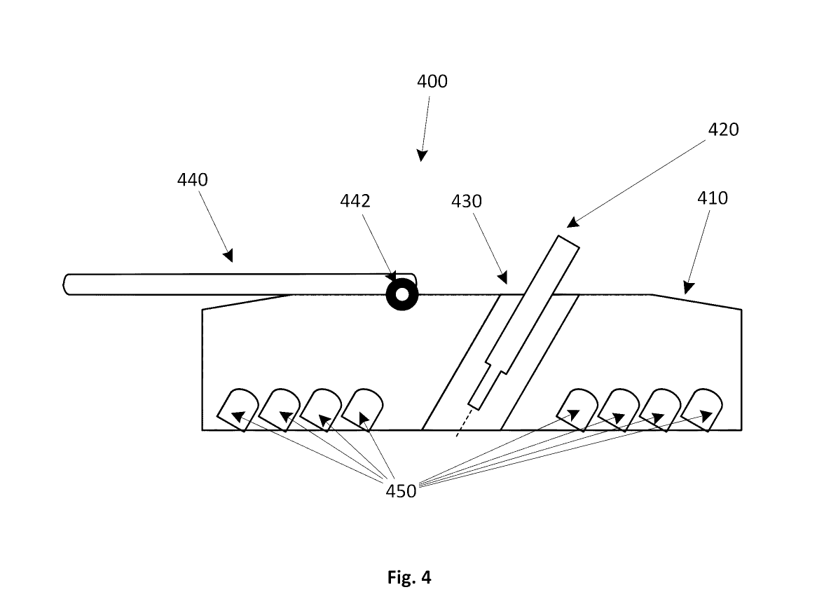

FIG. 4 is a side view of an exemplary, portable ultrasound imager 400 with graphical user interface 440 including a display screen and probe guide 430 together with a model of at least a portion of the imaged area, in accordance with an alternative embodiment of the disclosure provided herein. In the present embodiment, one or more transducers 450 are disposed proximal on either side of the probe guide 430 opposite thereto. One embodiment of the present configuration presents transducers 450 so as to be directed collinearly with needle 420 and probe guide 430.

In other embodiments, the user interface 440 can include a visual display screen (e.g., a LCD, touch display or similar display screen) which is housed in a frame and mechanically coupled to the body 410 of the device, e.g., at a hinged or pivoting coupling joint. Electrical connections between the body 410 and the user interface 440 may be carried out through ribbon connectors, pin connections or similar means 442. The angle of the display screen or interface 440 may thus be tilted with respect to the body 410 at a variety of angles to suit usage and viewing by a user of the device.

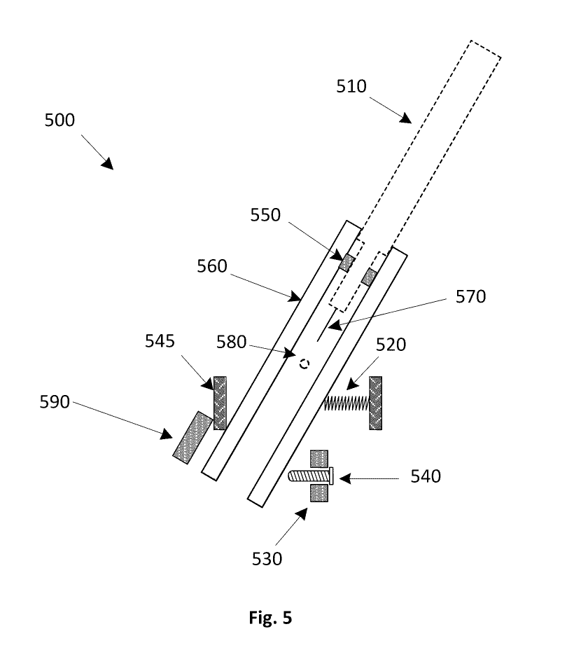

FIG. 5 illustrates an exemplary probe imaging and guidance mechanism 500 with a rotational degree of freedom, in accordance with some embodiments of the disclosure provided herein. FIG. 5 illustrates generally an example of a probe guide 530 and related apparatus, such as can be included in the examples of FIGS. 1-4 or other embodiments covered by this disclosure.

In one or more embodiments, a replaceable or removable insert, such as a seal 550, can be positioned along or within a portion of the probe guide 560. This serves to isolate a sterile portion of a probe assembly 510, such as a needle or catheter tip 570, from surrounding non-sterile portions of an assembly. The seal may be adhesively coated, or retained such as using a clamp, or an interference fit, or using one or more detents included as a portion of the probe guide 530.

In an example, the angle of the probe guide 530 can be adjusted or positioned, either manually by the user, or automatically, such as to provide a desired or specified probe insertion angle. For example, one or more of a setscrew 540, or a spring portion 520 can be used to pivot a channel of the probe guide, such as pivoting around a pin 580 in probe guide 560, or pivoting around another hinge or similar portion of the probe guide 560. In an example, the setscrew 540 can be retained by a threaded block 530, such as manually adjusted or driven by a mechanical actuator to allow automatic or semi-automatic rotation of the probe guide 560 about the pin 580.

One or more stops, such as a stop 545 can constrain the angular movement of probe guide 560 within a desired range of possible angular positions. In an example, a ball-and-spring apparatus and detents can be used, such as to allow a user to manually position the probe guide 560 in a desired angular position, with the detents indexing the probe guide 560 to specified angles, such as offset from each other by a specified angular increment.

In some embodiments, a piezoelectric element such as located nearby an opening (e.g., nearby an exit port of the probe guide 560), can be used to automatically measure the angle of the probe guide 560, or to provide feedback for automatic probe guide angular control. An initial distance between the center of a piezoelectric element and the opening of the probe guide can be measured before repositioning to provide a frame of reference or baseline, and thus the position of the opening can be tracked via a deviation from the frame of reference or baseline.

The angle of insertion of a probe (e.g., a needle) may be determined manually or via a processing circuit (e.g., a computer), such as based on information provided via the piezoelectric element. In this manner, depending on the depth of the probe assembly 510 within the guide 560, the angle of the probe guide 560 can be controlled such as to provide a desired final depth for the needle 570.

For example, a location of a needle 570 or catheter tip can be tracked, such as using a piezoelectric technique separate from the angular position measurement. Other techniques for tracking the probe assembly 510 position, or needle 570 position, can include using optical, magnetic techniques, or strain gauge. For example, one or more reference markings can be provided on a portion of the probe assembly 510 that can be visible within or at an entry port of the guide 560 (e.g., a ruler or scale can be imprinted on the probe assembly 510, such as visible to the user during insertion). In another embodiment the force of the needle 570 through the probe guide 560 can be sensed with a pressure sensor or strain gauge or can turn a gear through a gear mechanism. These approaches can be used to provide an estimate of the distance traveled by the needle 570 through the probe guide 560 and therefore an estimate of the location of the needle end.

In an example, a piezoelectric actuator can be coupled to the needle 570, or another portion of the probe assembly 510. As the probe is inserted into tissue of the subject, one or more techniques can then be used to track the probe tip location, such as via exciting the probe at a known frequency or at a known range of frequencies using the actuator, and locating the probe tip using, for example, color Doppler ultrasound techniques. In this way, information about the needle 570 location, within a subject, can be overlaid or otherwise displayed along with other anatomical information, such as to aid a user in positioning the probe tip at a desired location. In another embodiment, the probe can be magnetized and magnetic tracking can be used to determine the location of the probe.

In the above examples and others, a marking or pinching apparatus can be used in addition to or instead of the probe assembly 510, such as to pinch (e.g., discolor) or mark tissue at a insertion site, such as using the path provided by the probe guide 560. Such markings or discolorations can be later used by the practitioner to aid in inserting or guiding the probe during a puncture procedure. In an example, a template or patch can be deposited or adhered onto a site of the subject, such as at or near a location of a desired probe insertion site, such as after locating bone or other anatomical features using the hand-held ultrasonic apparatus of the above examples, or using apparatus or techniques of one or more other examples.

In an aspect, one or more portions of the rotational guide apparatus 500 can be separate from the hand-held ultrasonic assembly of FIGS. 1-4, or as shown and described in other examples. In such an example, the probe tip location can still be tracked using the hand-held apparatus, such as using the piezoelectric or other techniques discussed above. In an example, the hand-held apparatus can be used to mark or otherwise identify an insertion site for the probe, and a separate probe guide apparatus, such as shown in FIG. 4, can be used for insertion of the probe at a desired or specified angle.

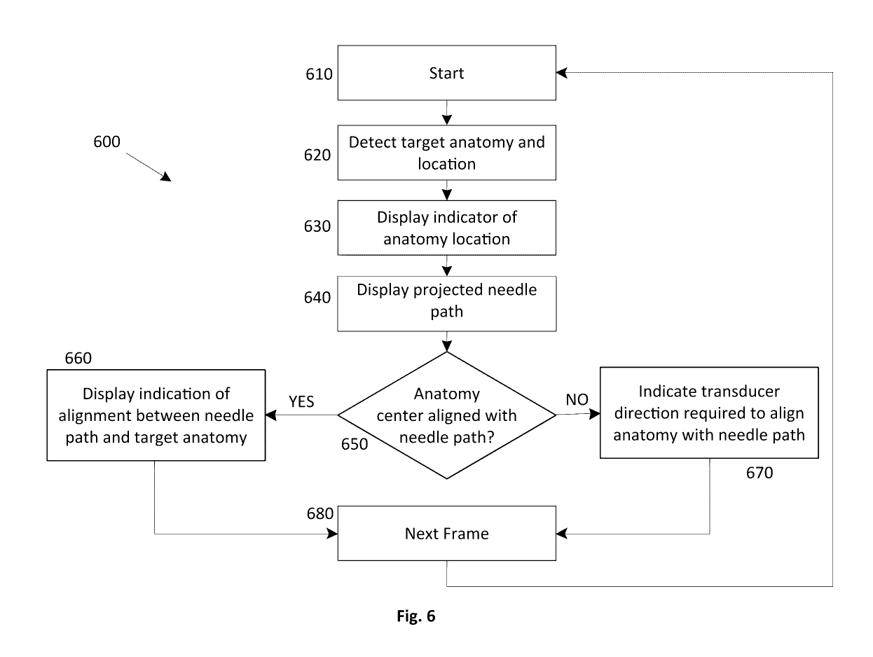

FIG. 6 is a flowchart 600 of an illustrative process for directing a probe in a fixed guide to a predetermined, anatomical location based at least in part on ultrasonic imaging, in accordance with some embodiments of the disclosure provided herein. The process described in the present embodiment utilizes one of the previously described methods for automated anatomical identification.

The process begins at 610 by detecting a prospective target anatomy location 620 relative to the imaging device. A display indicator of target anatomy 630 is presented on a GUI or similar interface. An abstraction of the ideal needle path is then portrayed on the display of the GUI 640. In one embodiment, the needle path is a predetermined, fixed needle path such as may be dictated from a needle guide with a fixed position and angle. A determination is then made by an arbiter or similar device to decide whether the target anatomy is centered within the needle path 650.

If so, an indicator of alignment between the needle path and target anatomy is displayed 660 on the display of the GUI. If non-alignment has been determined, a directional indicator is displayed depicting motion necessary for the ultrasonic device to be centered on the target anatomy 670, the details of which will be discussed in greater detail later in the application. Pursuant to real-time update imaging, next frame 680 loops the process to ensure accuracy.

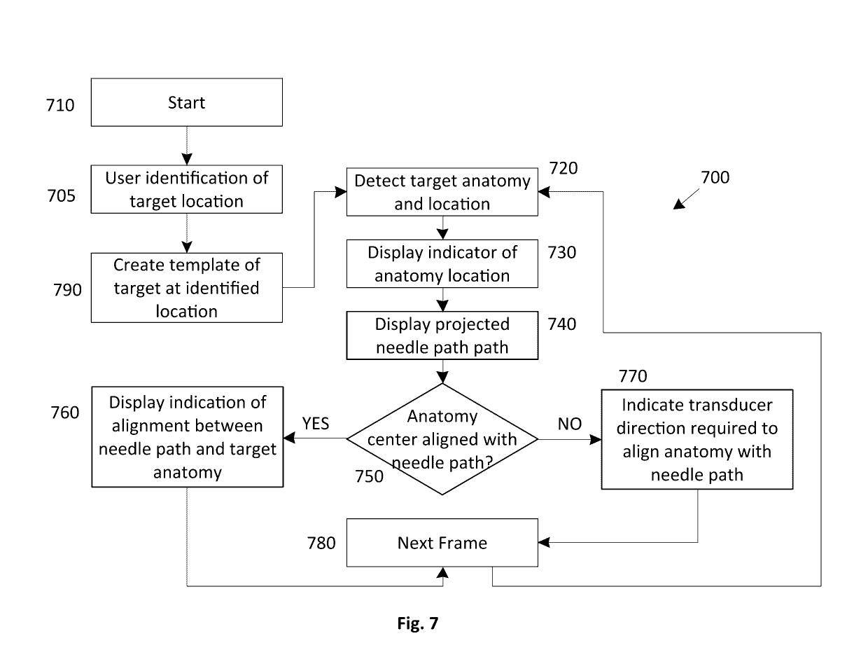

FIG. 7 is a flowchart 700 of an illustrative process of directing a probe in a fixed guide to a user-identified anatomical location based at least in part on ultrasonic imaging, in accordance with some embodiments of the disclosure provided herein. Here, the process begins at 705 by the identification of target anatomy and procedure location via GUI or other input device. Ultrasonic device creates a template of the target location and surrounding area 790. A template of the target location can be a sampling of image intensities at grid points surrounding the location identified by the user 705, or it could be some parameterized version of the local image region. For example the template could comprise the edge positions of the anatomical feature after performing an edge extraction routine, such as those known to those skilled in the art of image processing—i.e. Laplacian of a Gaussian filter.

Ultrasonic device then detects the template location within the current image 720. Detection of a template location within the current image can be achieved through a variety of methods such as those described above—e.g. normalized cross-correlation, shape models, or Hough transforms. A display indicator of target anatomy 730 is presented on a GUI or similar. An abstraction of the ideal needle path is then portrayed on the display of the GUI 740. A determination is then made by an arbiter or similar device to decide whether the target anatomy is centered within the needle path 750.

If so, an indicator of alignment between the needle path and target anatomy is displayed 760 on the display of the GUI. If non-alignment has been determined, a directional indicator is displayed depicting motion necessary for the ultrasonic device to be centered on the target anatomy 770, the details of which will be discussed in greater detail later in the application. Pursuant to real-time update imaging, next frame 780 loops the process to ensure accuracy.

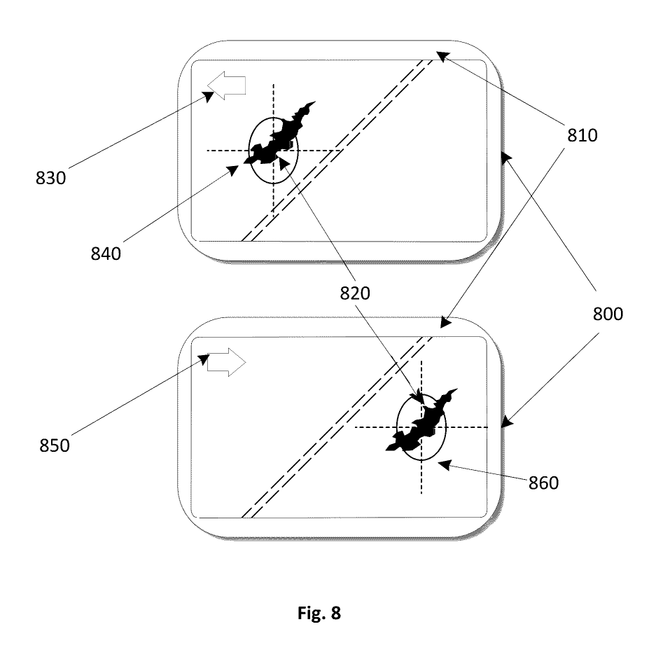

FIG. 8 depicts an exemplary graphical user interface (GUI) 800 demonstrating probe directional location feedback and overlaid ultrasound image of target anatomy 820, in accordance with some embodiments of the disclosure provided herein. The user interface can be mostly carried out using a visual screen and input/output actuators, sensors and similar elements. An underlying hardware, software and firmware system may be used to support the operation of the GUI, including a processor executing an operating system (e.g., Linux or an embedded software system). Indications can be provided by indicator symbols 830, 850 on the display screen of the GUI 800 and can indicate the direction by which the ultrasound transducer needs to translate in order for the target anatomy to align with the prospective needle path 810.

GUI indicators can indicate a motion of the ultrasound transducer that could include translation (as shown), compression, or rotation. In one or more embodiments mid-line indicators 840, 860 convey relative position of the ultrasonic device relative to the loaded template depicting target anatomy 820. That is, while the device may be surveyed over the patient anatomy, the GUI image may remain somewhat static (within the confines of the template). Instead, the mid-line indicators 840, 860 move in response to physical displacement of the ultrasonic device and relative to the depicted target anatomy 820. In an aspect, a practitioner moves the imaging head of the device over the skin of the patient, e.g., above the patient's spine, while observing the graphical output of the display screen of the device so as to determine the location of the spine, its vertebrae and other anatomical structures, and so as to determine the location into which a needle or probe are inserted relative to said spine and vertebrae. In one or more embodiments the mid-line indicators can be combined with an indication of the depth of the target anatomy, such depth can be automatically displayed alongside of the mid-line indicator.

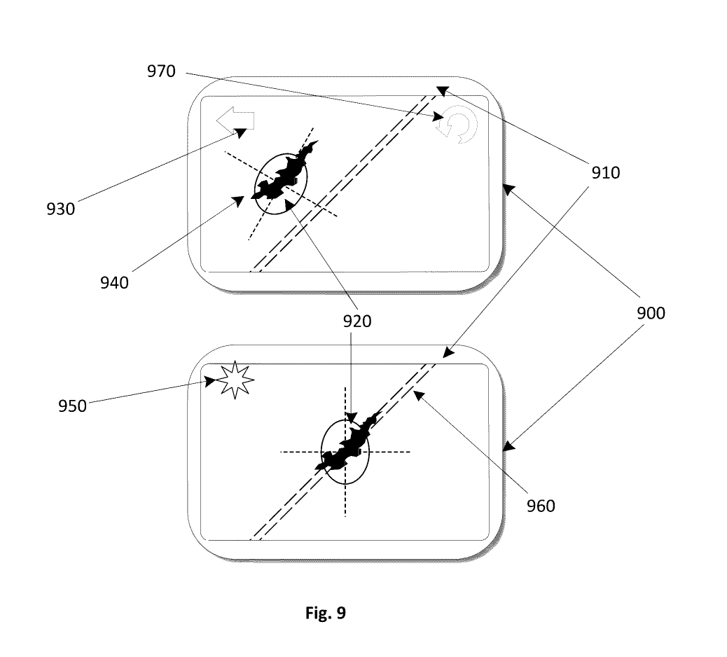

FIG. 9 depicts an exemplary graphical user interface (GUI) 900 demonstrating probe rotational disposition and directional feedback and overlaid ultrasound image of target anatomy 920, in accordance with some embodiments of the disclosure provided herein. Indications are provided by indicator symbols 930, 950, 970 on the display screen of the GUIs 900.

Indicator symbol 930 designates the direction by which the ultrasound transducer needs to translate in order for the target anatomy to align with the prospective needle path 910. As discussed, GUI indicators can designate necessary motion of the ultrasound transducer comprising translation (as shown), compression, or rotation. Indicator symbol 950 denotes that no translation is necessary and the prospective needle path 910 is aligned with the target anatomy 920.

Indicator symbol 970 designates a rotational direction by which the ultrasound transducer needs to translate in order for the target anatomy to align with the prospective needle path 910. In some embodiments indicator symbols (e.g., 930, 950) denote both magnitude and direction. For example, a larger necessary translation might be designated by longer arrow or indicator. In the present embodiment mid-line indicators 940, 960 convey relative disposition of the ultrasonic device relative to the loaded template depicting target anatomy 920.

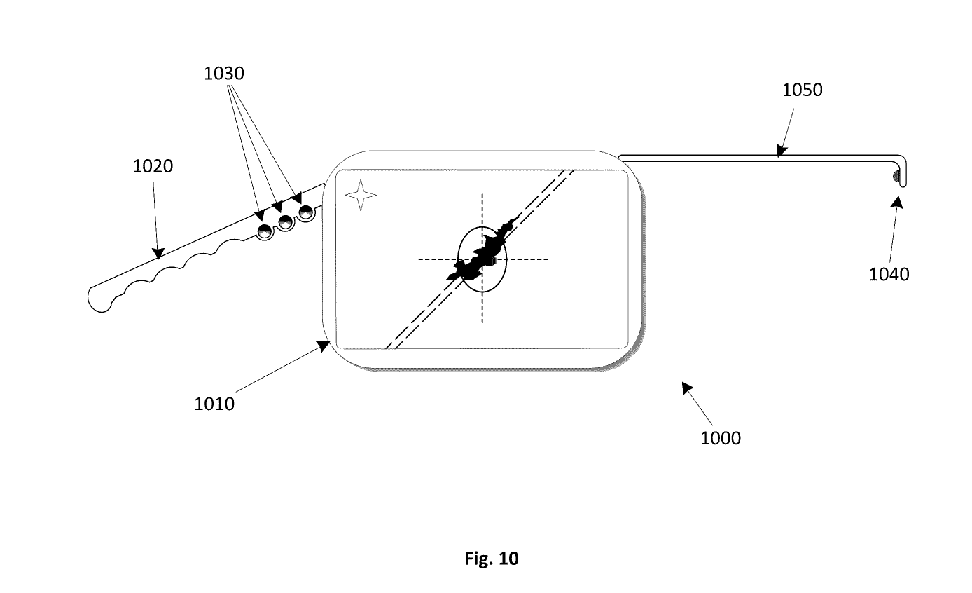

FIG. 10 is a top-down view of a portable ultrasound imager device 1000 with display/graphical user interface 1010 feedback depicting exemplary probe insertion and guidance thereto, in accordance with some embodiments of the disclosure provided herein. In the present embodiment, ultrasound system 1000 performs similarly to what has been indicated previously. Whereas, instead of assuming a fixed needle path, the needle path is not fixed. The system detects the target anatomy and also suggests an ideal needle path.

The system further detects the actual needle in the image and indicates a change in position required to align the actual needle path with the suggested needle path. In one embodiment, needle detection is performed by an optical detection system 1040, e.g., optical camera, laser positioning device, etc. However, in other embodiments, this may be performed via attached motion sensing, ultrasonic array phasing or any other suitable method.

Handle 1020 provides a convenient way to operate the ultrasonic imaging device 1000. Handle comprises buttons 1030 to provide access to templates and target anatomy selection, since presumably the user other hand will be occupied manipulating a needle for insertion. Alternatively, the user can make target anatomy selections via interaction with a touchscreen interface. Extension 1050 roughly defines the area to be displayed on display 1010.

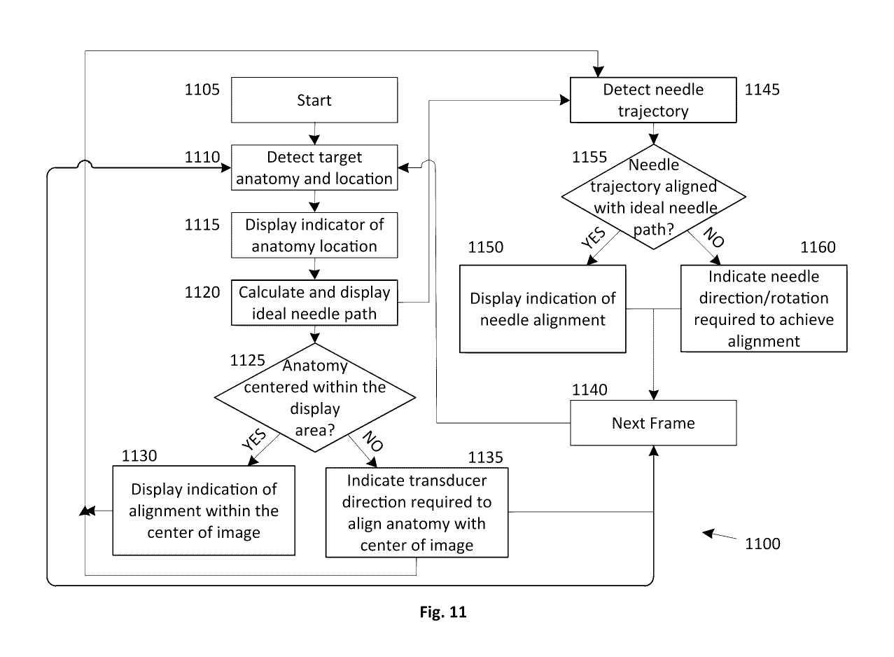

FIG. 11 is a flowchart 1100 of an exemplary procedure for directing a probe without a fixed angle probe guide to a detected anatomical feature based at least in part on a generated ultrasonic image, in accordance with some embodiments of the disclosure provided herein. The process described in the present embodiment utilizes one of the previously described methods for automated anatomical identification or user interaction-based detection.

The process beginning at 1105 detects the location of the prospective target anatomy 1110 relative to the ultrasound transducer. A display indicator of target anatomy 1115 is presented on a GUI or similar. An ideal needle path is calculated and an abstraction thereof is portrayed on the display of the GUI 1120. A determination is then made by an arbiter or similar device to decide whether the target anatomy is centered within the display area 1125.

In a preferred embodiment, the ideal needle is calculated to find a path through the image plane that most closely intersects with the location of the target anatomy. The calculated ideal needle path can be restricted to needle paths that exhibit one or more virtual or physical pivot points by which the angle of the needle can rotate. This method restricts the possible needle paths through the image plane by which the ultrasound system can select during the calculation. Alternatively the suggested needle paths can be restricted to more than one virtual pivot point, but these virtual pivot points are restricted within a particular area or volume. For example, the virtual pivot points would be restricted to a region superficial to the skin surface and adjacent to the ultrasound transducer. This restriction may be used because an actual pivot point cannot exist below the skin or inside of the ultrasound system.

A determination is then made by an arbiter or similar device to decide whether the target anatomy is centered within the display area 1125.

If so, an indicator of alignment of target anatomy and image center is displayed 1130 on the display of the GUI. If non-alignment has been determined, a directional indicator is displayed depicting motion necessary for the ultrasonic device to be centered on the target anatomy 1135. Pursuant to real-time update imaging, next frame loops the process to ensure accuracy 1140.

Alternatively after 1120, a calculated prospective needle path is depicted 1145, if the image in sufficiently centered. A determination is then made by an arbiter or similar device to decide whether the calculated needle trajectory is centered within the ideal needle path 1155.

If so, an indicator of needle alignment is displayed on the display of the GUI 1150. If non-alignment has been determined, a directional/rotational indicator is displayed depicting motion necessary for the needle to be centered on the target anatomy 1160. Pursuant to real-time update imaging, next frame 1140 loops the process to ensure accuracy.

In another embodiment, the calculation and display of an ideal needle path 1120 is instead user selectable. In this embodiment, multiple possible needle paths are displayed to the user via a graphical user interface, and the user can select which needle path they desire, for example via a touchscreen interface user input selection. The present inventors recognize that this embodiment may be particularly useful when the target anatomy does not exactly correspond to the desired placement of the needle. For example, in a nerve blockade injection, the target anatomy could be considered to be an easily recognizable blood vessel. However, the desired placement of the needle, which is the nerve bundle, is adjacent to the blood vessel. With the embodiment of a user selectable needle path, the user can select a needle path that intersects with the expected location of the nerve bundle rather than the target anatomy blood vessel.

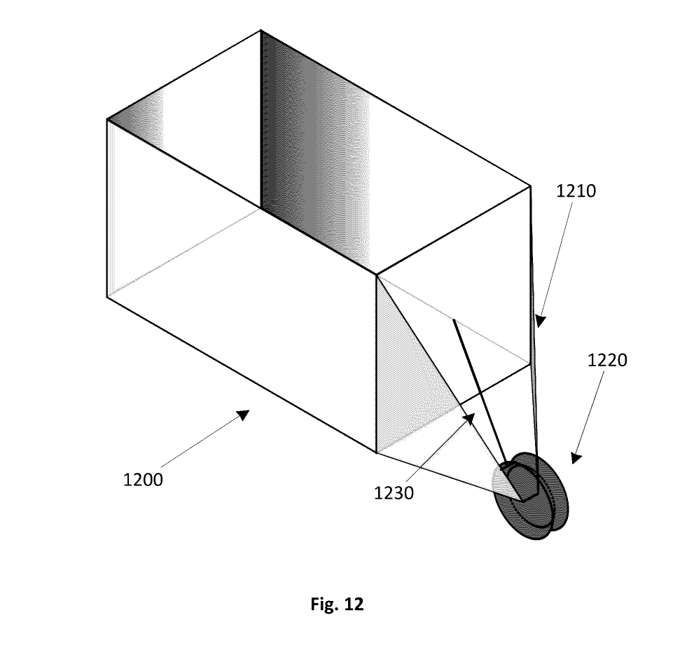

FIGS. 12-14 represent views of exemplary embodiments of the present system; as such, common identifiers are used for discussion thereof.

FIG. 12 shows an isometric view of an exemplary virtual axis probe guide 1200 rotating about a fixed pivot axis in the image plane for use in device assisted probe guidance.

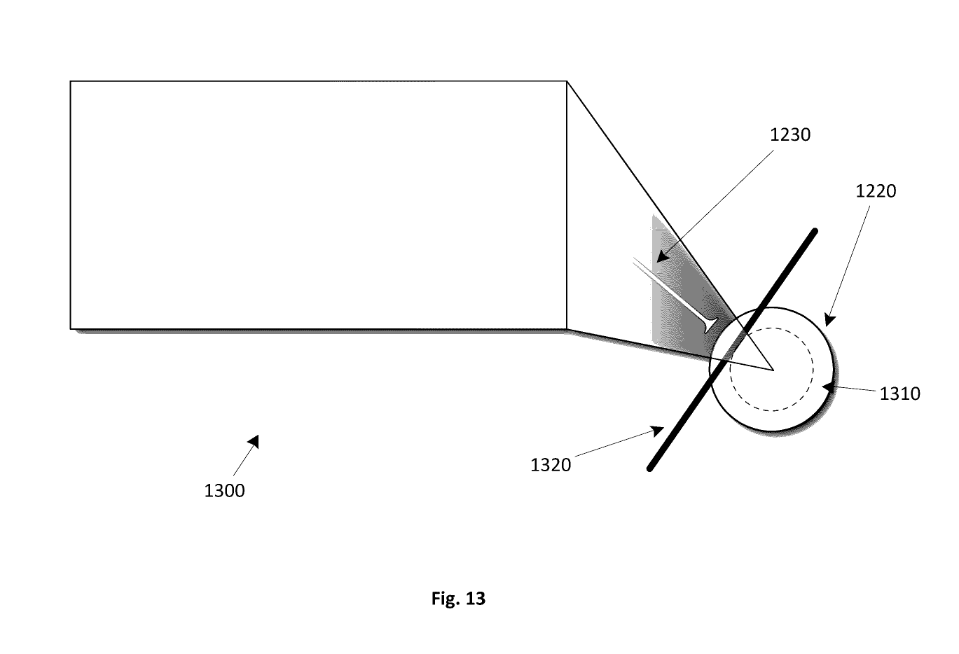

FIG. 13 illustrates a side view of an exemplary virtual axis probe guide 1300 rotating about a fixed pivot axis in the image plane for use in device assisted probe guidance.

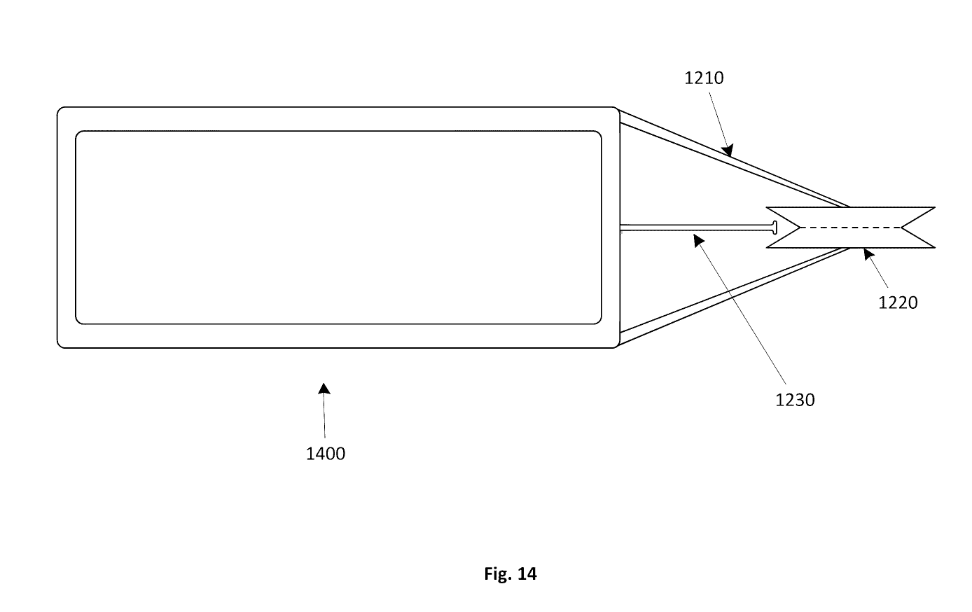

FIG. 14 illustrates a top-down view of an exemplary virtual axis probe guide 1400 rotating about a fixed pivot axis in the image plane for use device assisted guidance, in accordance with some embodiments of the disclosure provided herein.

A needle guide that restricts needle path to in-plane but within the image plane, the guide allows rotation about a pivot axis in order to access different areas within the image plane. Probe guide body 1200, 1300, and 1400 comprise four facing sides and brackets 1210, which secure guide spool 1220. While the present embodiment specifies four facing sides, any number of facing sides are acceptable as long as the guide spool 1220 is appropriately secured to the ultrasound system near the transducer. In the present embodiment, guide spool 1220 is cylindrical or circular to restrict motion of the probe out of the image plane but to allow rotation of the needle about a pivot point. Other shapes, such as elliptical, are not beyond the scope of the present invention.

The probe guide body 1200, 1300, and 1400 has a mechanism to force the probe 1220 to make physical contact against the diameter minimus 1310 spool unit 1220 and thereby retain the pivot point. This compression mechanism 1230 can be a physical spring or frictional force mechanism or it could be a magnetic force applied from the spindle unit (spool guide 1220). The friction force mechanism could be materials that physically interferes with the needle, but has low durometer (hardness or stiffness) so that it is compliant when the needle angle is adjusted.

The physical pivot point can be adjustable. Adjustment can be achieved via a latch, motor, or other similar mechanism. The physical pivot could be adaptively adjustable by the ultrasound system so that the pivot is adjusted for optimal needle approach. In this instance, the physical pivot would be electronically connected to the ultrasound system and an electronic motor mechanism could adjust the pivot based on calculations of the target location and ideal needle path.

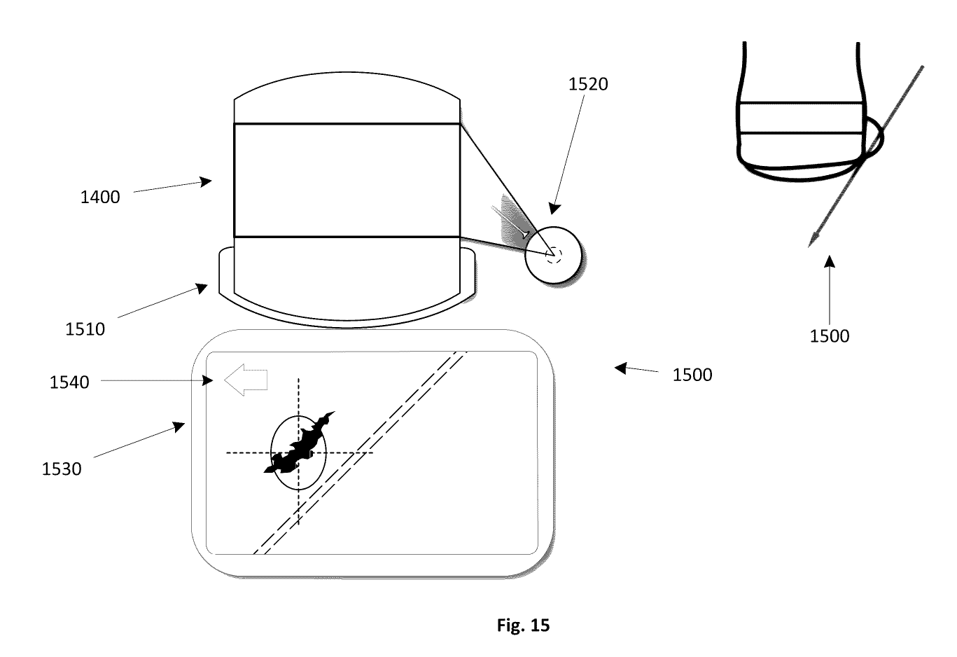

FIG. 15 is simplified side view 1500 of an exemplary probe guide 1400. The guide comprises a pivot axis 1520 in the image plane juxtaposed to a corresponding graphical user interface output 1530 with virtual state and disposition, in accordance with some embodiments of the disclosure provided herein. It should be noted that the juxtaposition of GUI 1530 is demonstrative of positioning the image plane and corresponding graphical display.

In one or more embodiments probe guide 1400 is sleeved down upon ultrasonic transducer array 1510. The present inventors recognize that the needle guide can also be integrated into the physical device housing or it could be a separate part that is sleeved down the ultrasonic transducer array. Furthermore, the present inventors recognize that the needle guide can be configured such that the needle is placed between the pivot point and the ultrasonic device or on the outside of both the pivot point and the ultrasonic device.

In practice, a target is aligned in the image to a location that is accessible by the needle through the needle guide. Ideal needle angle is indicated on GUI 1530. Relative configuration depicted in FIG. 15 illustrates a translational misalignment of the ideal needle path, which is denoted by in indicator symbol 1540.

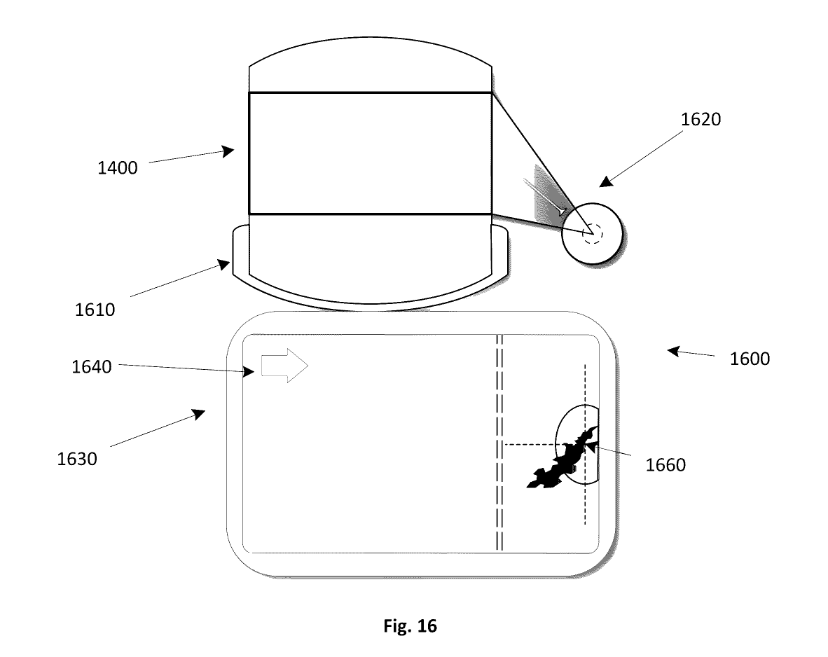

FIG. 16 is a graphical abstraction 1600 of a side view of an exemplary probe guide 1400 pivot axis. The guide comprises a pivot axis 1620 in the image plane juxtaposed to a corresponding graphical user interface output 1630 with virtual state and disposition, in accordance with some embodiments of the disclosure provided herein. It should be noted that the juxtaposition of GUI 1630 is demonstrative of positioning the image plane and corresponding graphical display.

As described, a target is aligned in the image plane to a location that is accessible by the needle through the needle guide. Target anatomy is identified using methods described above and indicated in the GUI 1630 with the indicator 1660. Ideal needle angle is further indicated on GUI 1630 as calculated by the ultrasound system using methods described above. The ideal needle angle is restricted to those obtainable assuming the virtual pivot point of the probe guide 1620 to achieve a path that is closest to an intersection with the target anatomy indicator 1660. Relative configuration depicted in FIG. 16 illustrates a target anatomy that is not accessible by a needle path—i.e. the needle path indicator does not intersect with the target anatomy indicator 1660. The translational indicator 1640 indicates a direction by which the ultrasound transducer needs to be translated in or order to better align the target anatomy indicator 1660 within the image plane to be accessible by the probe as restricted by the virtual pivot of the probe guide 1620.

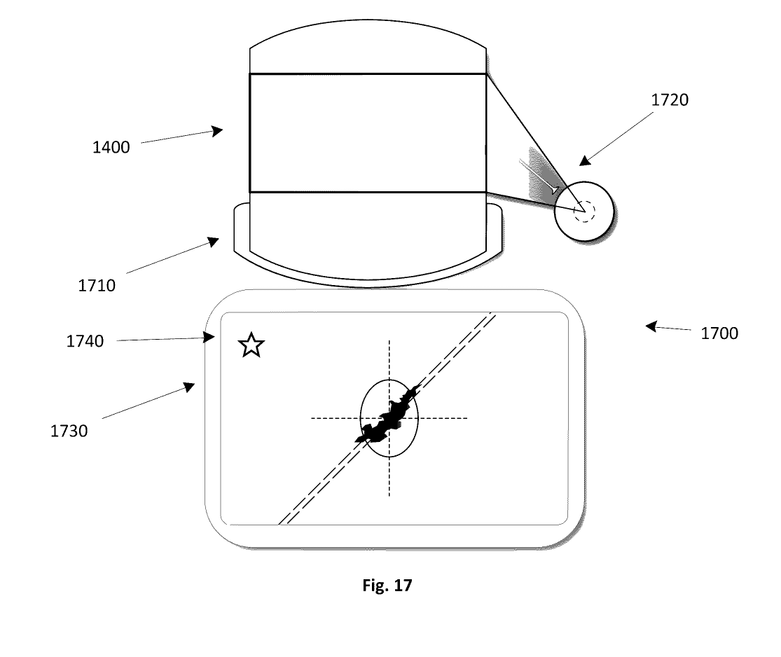

FIG. 17 is a graphical abstraction 1700 of a side view of an exemplary probe guide 1400 about a fixed pivot axis. The guide comprises a pivot axis 1720 in the image plane juxtaposed to a corresponding graphical user interface output 1730 with virtual state and disposition, in accordance with some embodiments of the disclosure provided herein. It should be noted that the juxtaposition of GUI 1730 is demonstrative of positioning the image plane and corresponding graphical display.

As described, the target is aligned in the image plane to a location that is accessible by the needle through the needle guide. Ideal needle angle is indicated on GUI 1730. Relative configuration depicted in FIG. 17 illustrates a translational alignment of the ideal needle path, denoted by an indicator symbol 1740.

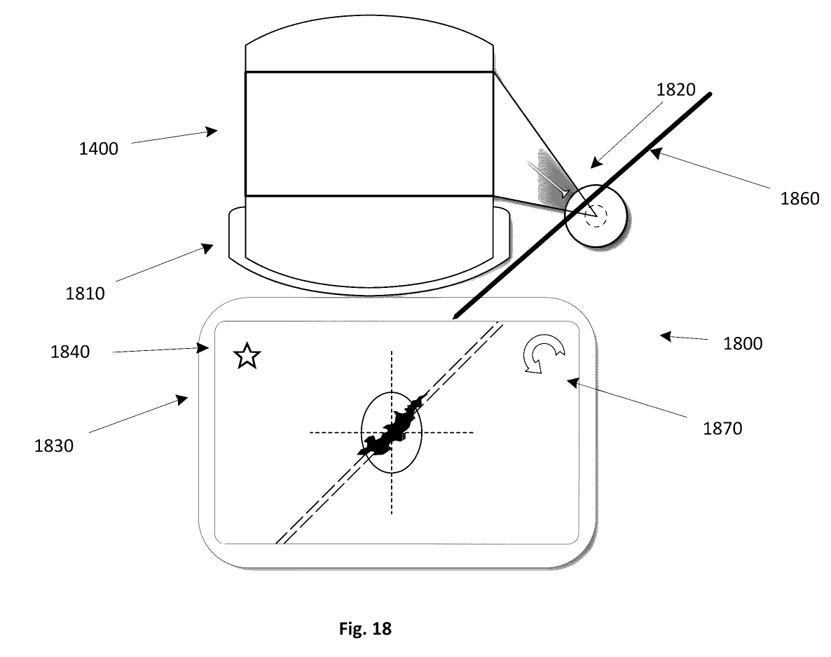

FIG. 18 is a graphical abstraction 1800 of a side view of an exemplary virtual probe guide 1400 about a fixed pivot axis. The guide comprises a pivot axis 1820 in the image plane juxtaposed to a corresponding graphical user interface output 1830 with virtual state and disposition, in accordance with some embodiments of the disclosure provided herein.

As described, the target is aligned in the image plane to a location that is accessible by the needle through the needle guide. In practice, needle 1860 angle is adjusted until it is calculated to correlate to intercept ideal needle path coaxially to reach target. Once proper translation is achieved to bring the target anatomy indicator in a portion of the image plane accessible by the needle 1860 through the needle guide 1820, indicator symbol 1840 is displayed and ideal needle path is determined. The actual needle angle is calculated by the ultrasound system. As described, there is a rotation required to bring the needle coincident to the ideal needle path indicator. As such, a needle rotation indicator 1870 is displayed to orient the user as to the needle angle adjustment required to place the needle on the ideal needle path.

Needle 1860 path is now restricted by needle guide 1820 to only two degrees of freedom: needle 1860 advancement and rotational angle. Ideal needle angle is indicated on GUI 1830. Relative configuration depicted in FIG. 18 illustrates a rotational misalignment of the ideal needle path, denoted by counter clockwise indicator symbol 1870.

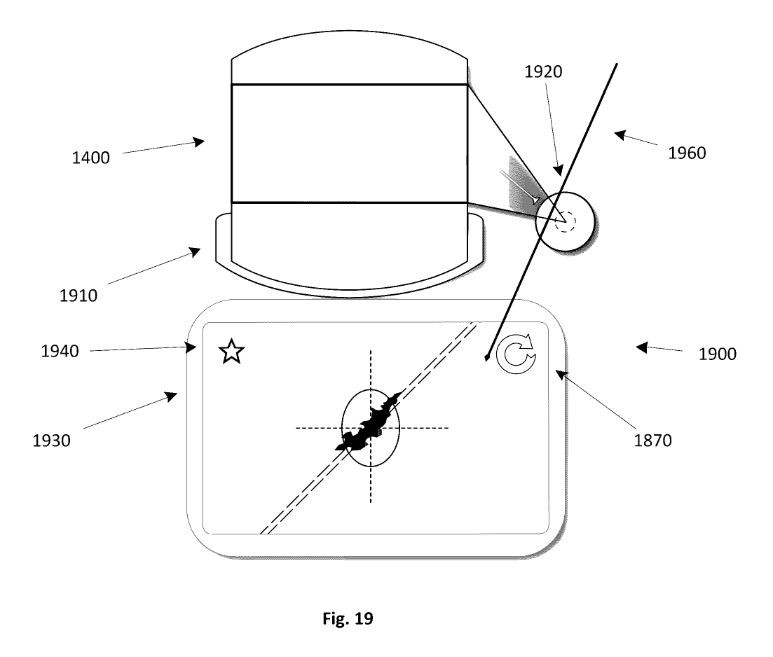

FIG. 19 is a graphical abstraction 1800 of a side view of an exemplary virtual probe guide 1400 rotating about a fixed pivot axis 1920 in the image plane juxtaposed to a corresponding graphical user interface output 1930 with virtual state and disposition, in accordance with some embodiments of the disclosure provided herein.

As described, the target is aligned in the image plane to a location that is accessible by the needle through the needle guide. In practice, needle 1960 angle is adjusted until it is calculated to correlate to intercept ideal needle path coaxially to reach target. Once proper translation is achieved to bring the target anatomy indicator in a portion of the image plane accessible by the needle 1960 through the needle guide 1920, indicator symbol 1940 is displayed and ideal needle path is determined. The actual needle angle is calculated by the ultrasound system. As described, there is a rotation required to bring the needle coincident to the ideal needle path indicator. As such, a needle rotation indicator 1870 is displayed to orient the user as to the needle angle adjustment required to place the needle on the ideal needle path.

Needle 1960 path is now restricted by needle guide 1820 to only two degrees of freedom: needle 1960 advancement and rotational angle. Ideal needle angle is indicated on GUI 1930. Relative configuration depicted in FIG. 19 illustrates a rotational misalignment of the ideal needle path, denoted by clockwise indicator symbol 1970.

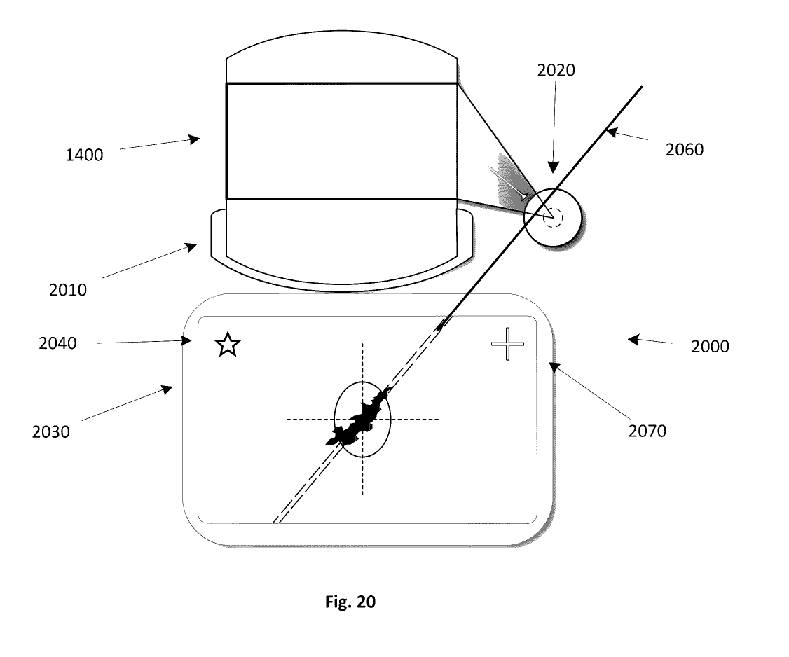

FIG. 20 is a graphical abstraction 2000 of a side view of an exemplary virtual probe guide 1400 rotating about a fixed pivot axis 2020 in the image plane juxtaposed to a corresponding graphical user interface output 2030 with virtual state and disposition, in accordance with some embodiments of the disclosure provided herein.

As described, the target is aligned in the image plane to a location that is accessible by the needle through the needle guide. In practice, needle 2060 angle is adjusted until it is calculated to correlate to intercept ideal needle path coaxially to reach target. Once proper translation is achieved to bring the target anatomy indicator in a portion of the image plane accessible by the needle 2060 through the needle guide 2020, indicator symbol 2040 is displayed and ideal needle path is determined. The actual needle angle is calculated by the ultrasound system. As described, the needle is coincident to the ideal needle path indicator. As such, an alignment indicator 2070 is displayed to convey to the user that the needle is along the ideal needle path.

Needle 2060 path is now restricted by needle guide 2020 to only two degrees of freedom: needle 2060 advancement and rotational angle. Ideal needle angle is indicated on GUI 2030. Relative configuration depicted in FIG. 20 illustrates a rotational alignment of the ideal needle path, denoted by cross indicator symbol 2070.

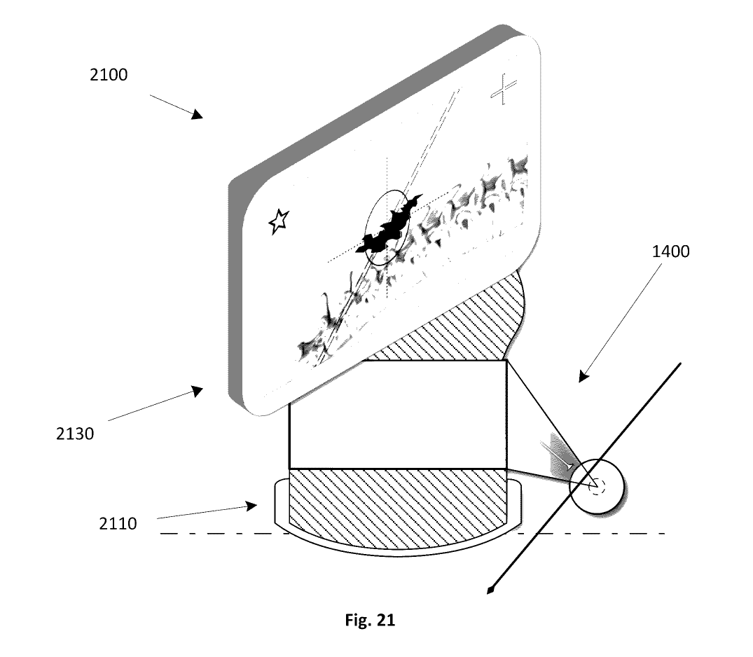

FIG. 21 illustrated an exemplary handheld ultrasound imager 2100 with graphical user interface 2130 feedback and non-affixed probe guide together with an automated detection of target anatomy and ideal needle path of at least a portion of the imaged area, in accordance with some embodiments of the disclosure provided herein. FIG. 21 demonstrates the handheld device with a virtual axis probe guide 1400 coupled to a transducer array 2110 with a GUI 2130 and automated guide. As described, the display is directly integrated into the transducer hand grip region without a cable attachment. It is recognized by the present inventors that this configuration has advantages of being more intuitive for the user as the display screen is in-line with the underlying anatomy that is being targeted by the probe.

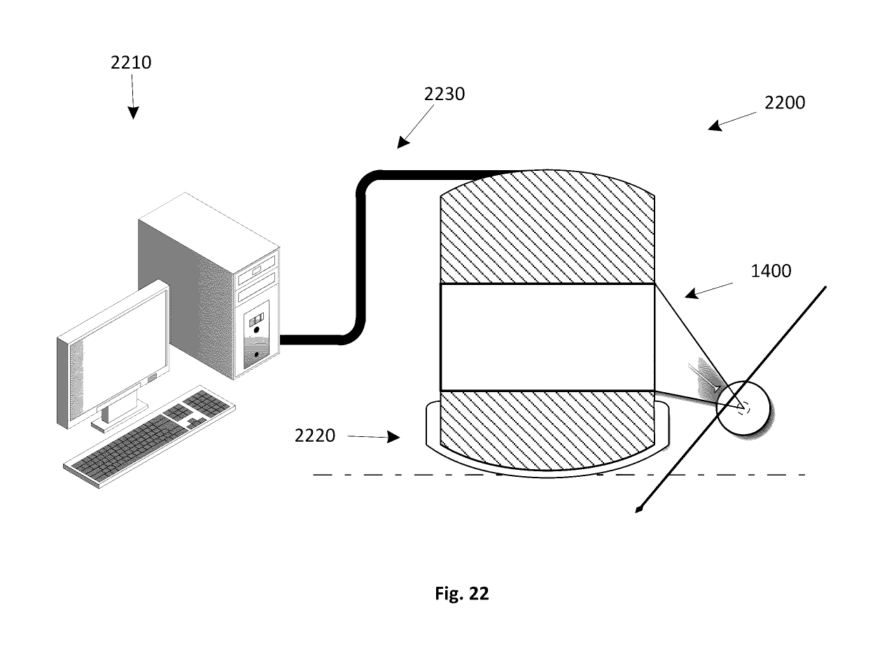

FIG. 22 illustrated an exemplary portable 2D ultrasound imager 2200 coupled to external computational unit 2210 via data communication 2230 and non-affixed probe guide 1400 together with an automated detection of target anatomy and ideal needle path of at least a portion of the imaged area, in accordance with some embodiments of the disclosure provided herein. FIG. 22 demonstrates the capacity portable device 2200 with a virtual axis probe guide 1400 coupled to a transducer array 2110 with a computational unit 2210.

Having thus described several aspects and embodiments of the technology of this application, it is to be appreciated that various alterations, modifications, and improvements will readily occur to those of ordinary skill in the art. Such alterations, modifications, and improvements are intended to be within the spirit and scope of the technology described in the application. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein.

Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments may be implemented in any of numerous ways. One or more aspects and embodiments of the present application involving the performance of processes or methods may utilize program instructions executable by a device (e.g., a computer, a processor, or other device) to perform, or control performance of, the processes or methods.

In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above.

The computer readable medium or media may be transportable, such that the program or programs stored thereon may be loaded onto one or more different computers or other processors to implement various ones of the aspects described above. In some embodiments, computer readable media may be non-transitory media.

An illustrative implementation of a computer system 2210 that may be used in connection with any of the embodiments of the disclosure provided herein. The computer system 2210 may include one or more processors 104 and one or more articles of manufacture that comprise non-transitory computer-readable storage media (e.g., memory 116 and one or more non-volatile storage media). The processor 104 may control writing data to and reading data from the memory 116 and the non-volatile storage device in any suitable manner, as the aspects of the disclosure provided herein are not limited in this respect. To perform any of the functionality described herein, the processor 104 may execute one or more processor-executable instructions stored in one or more non-transitory computer-readable storage media (e.g., the memory 116), which may serve as non-transitory computer-readable storage media storing processor-executable instructions for execution by the processor 104.

The terms “program” or “software” are used herein in a generic sense to refer to any type of computer code or set of computer-executable instructions that may be employed to program a computer or other processor to implement various aspects as described above. Additionally, it should be appreciated that according to one aspect, one or more computer programs that when executed perform methods of the present application need not reside on a single computer or processor, but may be distributed in a modular fashion among a number of different computers or processors to implement various aspects of the present application.

Computer-executable instructions may be in many forms, such as program modules, executed by one or more computers or other devices. Generally, program modules include routines, programs, objects, components, data structures, etc. that performs particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

When implemented in software, the software code may be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smart phone or any other suitable portable or fixed electronic device.

Also, a computer may have one or more input and output devices. These devices can be used, among other things, to present a user interface. Examples of output devices that may be used to provide a user interface include printers or display screens for visual presentation of output and speakers or other sound generating devices for audible presentation of output. Examples of input devices that may be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks or wired networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

The present invention should therefore not be considered limited to the particular embodiments described above. Various modifications, equivalent processes, as well as numerous structures to which the present invention may be applicable, will be readily apparent to those skilled in the art to which the present invention is directed upon review of the present disclosure.

What is claimed is:

1. An ultrasound imaging method comprising:
in a probe guidance system comprising a processor and a probe guide having a specified path along which to insert a probe:
transmitting one or more ultrasound signals from one or more transducers in the probe guidance system;
obtaining ultrasound data generated based, at least in part, on one or more ultrasound signals from an imaged region of a subject;
selecting a target anatomy associated with the imaged region based at least in part on the generated ultrasound data;
displaying an ultrasound image of the subject at least in part by combining the ultrasound data and the selected target anatomy;
determining a location of the imaged region relative to the target anatomy and the one or more transducers; and
calculating a projected probe path of the probe, the projected probe path indicative of an actual path to be taken by the probe when the probe is inserted through the probe guide;
generating a graphic indicator including generating a visible representation of said projected probe path, the visible representation of the projected probe path displayed with respect to said target anatomy.

2. The method of claim 1, wherein said projected probe path includes a projected needle path.

3. The method of claim 1, further comprising providing feedback in a loop when the probe guidance system determines that the projected probe path and the target anatomy are not collinear.

4. The method of claim 3, further comprising displaying a directional indicator to indicate a direction to translate the one or more transducers to align the projected probe path with the target anatomy.

5. The method of claim 3, further comprising displaying a rotational indicator to indicate a motion necessary to align the projected probe path with the target anatomy.

6. The method of claim 1, further comprising calculating an ideal probe path, the ideal probe path coaxially intersecting the target anatomy.

7. The method of claim 6, further comprising restricting the ideal probe path to potential probe paths that exhibit one or more physical pivot points by which an angle of said probe guide can rotate.

8. The method of claim 6, further comprising restricting the ideal probe path to potential probe paths that exhibit one or more virtual pivot points by which an angle of said probe guide can rotate.

9. The method of claim 1, further comprising calculating and displaying one or more displayed needle paths on a graphical user interface and comprising a user selecting and executing one of the displayed needle paths via interaction with a graphical user interface.

10. A probe guidance system comprising:
a user interface having a display with one or more symbolic indicators;
one or more ultrasonic transducers of an ultrasonic imaging unit configured and adapted to transmit and receive signals based at least in part on a target anatomy;
a probe guide having a specified path along which to insert a probe;
a processor for (a) determining a location of the target anatomy relative to the ultrasound imaging system and (b) calculating a direction to translate or rotate the one or more transducers to align (x) a projected probe path of the probe, the projected probe path indicative of an actual path to be taken by the probe when the probe is inserted through the probe guide, with (y) the target anatomy.

11. The probe guidance system of claim 10 wherein the displayed symbolic indicator represents the direction for a user to translate or rotate the one or more transducers.

12. The probe guidance system of claim 10 wherein the probe guide provides a variable rotational orientation relative to a surface of a patient.

13. The probe guidance system of claim 13 further comprising an integrated, real-time needle detection device.

14. The probe guidance system of claim 13 wherein the integrated, real-time needle detection device is optical.

15. The probe guidance system of claim 14 wherein the integrated, real-time needle detection device includes a piezoelectric element.

16. The probe guidance system of claim 13 wherein the processor calculates a current probe angle and determines a probe angle adjustment needed to align the projected probe path with the target anatomy.

17. The probe guidance system of claim 10 wherein the display comprises a touch-pad adapted and configured to accept user input to identify said target anatomy.

18. The probe guidance system of claim 10 wherein at least a portion of the probe guide is rotatable about a pivot point.

19. The probe guidance system of claim 18, wherein the probe guide includes a guide spool that defines the specified path along which to insert the probe, the pivot point on the guide spool.

20. The probe guidance system of claim 19, further comprising a compression mechanism that contacts the guide spool to retain the guide spool at a desired orientation.
